SEQUENCE LISTING (1 sequence)

SEQ ID NO: 1 - DNA, 825 bp, Homo sapiens
  source 1..825: mol_type = genomic DNA
atggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagttccccctgcagtggtttggagtccctgccattctgaaaggctggtttgagcgagtgttcataggagagtttgcttacacttacgctgccatgtatgacaaaggacccttccggagtaagaaggcagtgctttccatcaccactggtggcagtggctccatgtactctctgcaagggatccacggggacatgaatgtcattctctggccaattcagagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatga

(19) United States
(12) Patent Application Publication
(10) Pub. No.: US 2026/0240816 A1
(43) Pub. Date: Aug. 20, 2026

(54) METHOD OF TREATING PRIMARY MITOCHONDRIAL DISEASE

(71) Applicant: Curome Biosciences Co., Ltd., Chungcheongbuk-do (KR)

(72) Inventors: Joo Seog Yoon, Gyeonggi-do (KR); Kang Sik Seo, Gyeonggi-do (KR); Min Jung Kwon, Gyeonggi-do (KR); Sung Je Moon, Gyeonggi-do (KR); Yoon Seon Yoo, Gyeonggi-do (KR); Soo Bin Yun, Gyeonggi-do (KR)

(21) Appl. No.: 19/053,780

(22) Filed: Feb. 14, 2025

(51) Int. Cl. A61K 31/416; A61K 31/343; A61K 31/352; A61P 3/00; A61P 25/28; A61P 27/02; C12Q 1/6883

(52) U.S. Cl. CPC A61K 31/416; A61K 31/343; A61K 31/352; A61P 3/00; A61P 25/28; A61P 27/02; C12Q 1/6883; C12Q 2600/106; C12Q 2600/118; C12Q 2600/156

(57) ABSTRACT

The present disclosure relates to a method of treating primary mitochondrial disease, comprising a 1,2-naphthoquinone derivative. The method of the present disclosure enables the effective treatment of primary mitochondrial disease.

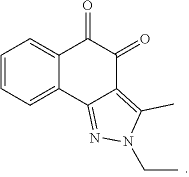 [SMILES: CCn1nc2c(c1C)C(=O)C(=O)c1ccccc1-2]

### SEQUENCE LISTING

This application contains references to amino acid sequences and/or nucleic acid sequences which have been submitted concurrently herewith as the sequence listing XML file entitled “000376us_SequenceListing.XML”, file size 2507 bytes, created on 14 Feb. 2025. The aforementioned sequence listing is hereby incorporated by reference in its entirety pursuant to 37 C.F.R. § 1.52(e)(5).

### FIELD

The present disclosure relates to a method of treating primary mitochondrial diseases, comprising a 1,2-naphthoquinone derivative.

### BACKGROUND

Mitochondria are essential cellular organelles that function as the cell's “powerhouse.” Defects in mitochondrial function due to genetic or environmental factors often lead to various severe diseases, particularly affecting organs with high energy demands, such as muscles and the brain. Mitochondrial diseases can be broadly classified into primary mitochondrial diseases (PMD) and secondary mitochondrial diseases (SMD). While SMD can be caused by genes unrelated to the function or production of OXPHOS (oxidative phosphorylation) proteins and are often associated with many hereditary non-mitochondrial disorders, PMD is defined as diseases that directly code for OXPHOS proteins or affect the production of complexes necessary for performing the OXPHOS process. Currently, methods for distinguishing PMD from SMD are known through next-generation sequencing. Diseases resulting from mitochondrial dysfunction may include abnormalities due to oxidative stress caused by mitochondrial membrane potential disruption, reactive oxygen species, or free radicals; dysfunction due to genetic factors such as mutations in nuclear DNA or mitochondrial DNA; and disorders resulting from defects in the OXPHOS function responsible for mitochondrial energy production. In particular, primary mitochondrial diseases caused by these factors include examples such as MELAS (mitochondrial encephalomyopathy, lactic acidosis, and stroke-like episodes) syndrome, Leigh syndrome, Kearns-Sayre syndrome, MERRF (Myoclonus Epilepsy with Ragged-Red Fibers), MIDD (Maternally Inherited Diabetes and Deafness), CPEO (Chronic Progressive External Ophthalmoplegia), LHON (Leber Hereditary Optic Neuropathy), and PMM (Primary Mitochondrial Myopathy). In the case of primary mitochondrial diseases, since they result from factors that affect the function and production of OXPHOS proteins, they can be effectively treated by restoring and/or activating the OXPHOS-electron transport process.

However, there have been no fundamental treatments for such mitochondrial dysfunction-related diseases, and only symptomatic relief through antioxidants, vitamins, and the like has been possible.

### SUMMARY

### Technical Problem

The inventors of the present disclosure have conducted extensive research to establish a treatment protocol aimed at enhancing the therapeutic efficacy and achieving favorable prognoses in subjects suffering from primary mitochondrial diseases. As a result, they have discovered for the first time that the administration of a pharmaceutical composition comprising a 1,2-naphthoquinone derivative can effectively treat primary mitochondrial diseases. Furthermore, it was found that the therapeutic efficacy is significantly enhanced in specific patient groups. This discovery not only improves the treatment efficiency for subjects with primary mitochondrial diseases but also allows for the selection of alternative treatment protocols for patient groups predicted to have poor prognoses, thereby completing the present disclosure.

Accordingly, one aspect of the present disclosure is directed to providing a method of treating primary mitochondrial diseases.

In another aspect of the present disclosure is directed to providing a method of selecting subjects for administration of 1,2-naphthoquinone derivative described herein.

In yet another aspect, the present disclosure provides a method of predicting prognosis following administration of 1,2-naphthoquinone derivative described herein.

### Solution to Problem

An aspect of the present disclosure provides a method of treating a primary mitochondrial disease in a subject in need thereof, the method comprising administering a pharmaceutically effective amount of a 1,2-naphthoquinone derivative.

The inventors have conducted extensive research to establish a treatment protocol aimed at improving therapeutic efficacy and achieving favorable outcomes in subjects suffering from primary mitochondrial diseases. As a result, the inventors have unexpectedly discovered that the administration of a pharmaceutical composition comprising a 1,2-naphthoquinone derivative effectively treats primary mitochondrial diseases. Furthermore, they have found, for the first time, that the therapeutic efficacy is significantly enhanced in specific patient populations. This discovery demonstrates that treatment efficiency can be improved for subjects with primary mitochondrial diseases and enables the selection of alternative treatment protocols for patient populations predicted to have poor prognoses.

The term “primary mitochondrial diseases” as used in the present disclosure may also be referred to as “primary mitochondrial disorders,” and refers to diseases caused by dysfunction within the mitochondrial electron transport chain (respiratory chain), an organelle responsible for energy production within cells (Wallace DC. Mitochondrial diseases in man and mouse. Science. 1999; 283:1482-8). More specifically, primary mitochondrial diseases are genetic disorders known or presumed to result from pathogenic mutations in genes encoding mitochondrial electron transport chain components and related proteins. The term “primary mitochondrial diseases” as used in the present disclosure may also be referred to as “primary mitochondrial disorders,” and refers to diseases caused by dysfunction within the mitochondrial electron transport chain (respiratory chain), an organelle responsible for energy production within cells (Wallace D C. Mitochondrial diseases in man and mouse. Science. 1999; 283:1482-8). More specifically, primary mitochondrial diseases are genetic disorders known or presumed to result from pathogenic mutations in genes encoding mitochondrial electron transport chain components and related proteins.

The mitochondrial electron transport chain is a critical pathway for aerobic metabolism, and tissues and organs that rely on aerobic metabolism are particularly susceptible to mitochondrial dysfunction. For example, primary mitochondrial diseases can affect any organ or tissue in the body that requires high levels of energy, such as the brain, heart, liver, and muscles, leading to a wide range of symptoms. Primary mitochondrial diseases can be fundamentally treated by restoring and/or activating the OXPHOS-electron transport process of the electron transport chain (respiratory chain).

As used herein, the term “treating” refers to any act of alleviating or favorably modifying the symptoms caused by primary mitochondrial diseases through the administration of the composition of the present disclosure. Specifically, “treating” in the context of the present disclosure may include, for example, a significant increase in the total ATP levels in the body of a subject suffering from primary mitochondrial diseases, or a significant decrease in total lactate and/or total ROS levels in the body as a result of administering the composition. The aforementioned “increase” or “decrease” refers to a change of at least 3%, 4%, 5%, 6%, 7%, 8%, 9%, 10%, 11%, 12%, 13%, 14%, 15%, 16%, 17%, 18%, 19%, or 20% in total ATP, lactate, and/or ROS levels in the subject after administration of the composition compared to the levels prior to administration. However, “treating” in the context of the present disclosure is not limited to changes in total ATP, lactate, and/or ROS levels as described above, but also encompasses any clinically recognized improvement in symptoms that may arise from the onset of primary mitochondrial diseases.

As used herein, the term “pharmaceutically effective amount” refers to an amount sufficient to achieve the efficacy or activity of the aforementioned 1,2-naphthoquinone derivative. Achieving the “efficacy or activity” as described above refers to inducing the changes defined under the “treating” of the present disclosure. Specifically, the term “pharmaceutically effective amount” encompasses any dosage capable of eliciting the therapeutic effect described herein.

The term “1,2-naphthoquinone derivative” of the present disclosure refers to a compound having a 1,2-naphthoquinone core structure. Specifically, the compound may further include a heterocyclic substituent as an additional component forming a ring with the carbon atoms at positions 3 and 4. The heteroatoms included in the heterocyclic substituent may be oxygen, nitrogen, or sulfur, and the heterocyclic substituent may contain one or more heteroatoms. The heterocyclic substituent may include one or more double bonds, and one or more C1-C4 straight-chain or branched-chain alkyl substituents may be additionally bonded to the atoms constituting the heterocyclic ring.

In one embodiment, the 1,2-naphthoquinone derivative of the present disclosure may be at least one compound selected from the group consisting of compounds represented by Chemical Formulae 1 to 3.

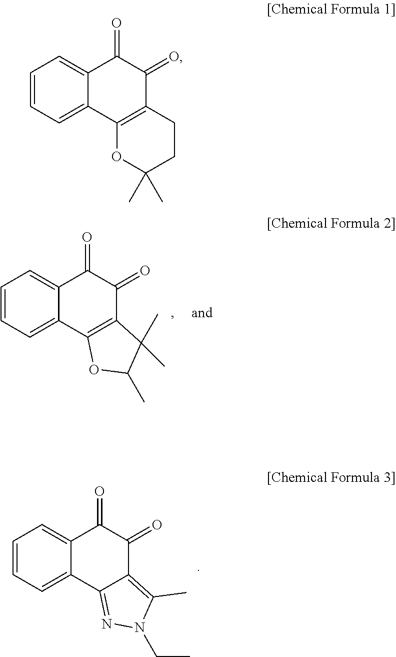 [SMILES: CC1(C)CCC2=C(O1)c1ccccc1C(=O)C2=O ; CC1OC2=C(C(=O)C(=O)c3ccccc32)C1(C)C ; "," ; "and" ; CCn1nc2c(c1C)C(=O)C(=O)c1ccccc1-2 ; "."]

The compound of Chemical Formula 1 is 2,2-dimethyl-3,4-dihydrobenzo[h]chromene-5,6-dione, which is known as Beta-Lapachone and may be referred to and described as HK-660S in the present disclosure.

The compound of Chemical Formula 2 is 2,3,3-trimethyl-2H-benzo[g][1]benzofuran-4,5-dione, which corresponds to a compound known as Dunnione.

The compound of Chemical Formula 3 corresponds to 3-methyl-2-ethylbenzo[g]indazole-4,5-dione.

In one embodiment, the 1,2-naphthoquinone derivative is administered to a subject in whom complex III, complex IV, and complex V of the mitochondrial electron transport chain function normally.

The electron transport chain (ETC), located in the inner mitochondrial membrane, is composed of protein complexes and ion channels. The mitochondrial electron transport chain consists of four protein complexes (complexes I-IV) and two electron carriers, ubiquinone (UQ) and cytochrome c (Cyt c). Complex I may be referred to as NADH-ubiquinone oxidoreductase, NADH-CoQ reductase, or NADH dehydrogenase. Complex II may be referred to as succinate dehydrogenase or succinate-CoQ reductase. Complex III may be referred to as cytochrome bc1 complex or CoQH2-cytochrome c reductase. Complex IV may be referred to as cytochrome c oxidase. The mitochondrial electron transport chain generates ATP, the primary energy storage molecule, through the electron transfer process mediated by complexes I-IV, ubiquinone, and cytochrome c. Each of complexes I-IV, which constitute the aforementioned electron transport chain, is a protein complex composed of multiple proteins, and functional impairments may occur due to mutations in the genes encoding these proteins or other forms of damage. When administered to a subject in whom at least complex III, complex IV, and complex V of the mitochondrial electron transport chain function normally, the composition of the present disclosure can effectively treat primary mitochondrial diseases and associated pathological conditions.

In one embodiment, the 1,2-naphthoquinone derivative is administered to a subject with impaired complex I of the mitochondrial electron transport chain.

The impairment of complex I, as described herein, includes both congenital and acquired impairments. More specifically, such impairments encompass congenital dysfunction due to mutations in genetic traits associated with complex I, acquired modifications, and functional inhibition caused by endogenous or exogenous factors that suppress the function of complex I. Diseases closely associated with complex I impairment include known primary mitochondrial diseases, such as MELAS syndrome, Leigh syndrome, Kearns-Sayre syndrome, MERRF, MIDD, CPEO, LHON, and PMM. The composition of the present disclosure can be suitably administered to a patient population in which primary mitochondrial disease has developed due to complex I impairment. The presence or absence of complex I impairment can be readily determined using methods well known in the art. More specifically, it can be identified through activity analysis assays for each mitochondrial complex. In particular, the impairment of complex I can be assessed by analyzing the binding of NADH to complex I and the conversion of ubiquinone (Q) to ubiquinol (QH2) to detect a significant decrease in activity.

For example, the activity of complex I can be analyzed using commercially available mitochondrial complex I activity assay kits (e.g., MAK359, Sigma-Aldrich) in accordance with the manufacturer's instructions.

In one embodiment, the 1,2-naphthoquinone derivative is administered to a subject having a genotype of CC or CT at position 609 of the NAD(P)H:quinone oxidoreductase 1 (NQO1) gene. The complete nucleotide sequence of the NQO1 gene is provided below, wherein nucleotide C at position 609 is indicated in bold and underlined.

ATGGTCGGCAGAAGAGCACTGATCGTACTGGCTCACTCAGAGAGGACG
TCCTTCAACTATGCCATGAAGGAGGCTGCTGCAGCGGCTTTGAAGAAGAAAGGAT
GGGAGGTGGTGGAGTCGGACCTCTATGCCATGAACTTCAATCCCATCATTTCCAG
AAAGGACATCACAGGTAAACTGAAGGACCCTGCGAACTTTCAGTATCCTGCCGAGT
CTGTTCTGGCTTATAAAGAAGGCCATCTGAGCCCAGATATTGTGGCTGAACAAAAG
AAGCTGGAAGCCGCAGACCTTGTGATATTCCAGTTCCCCCTGCAGTGGTTTGGAG
TCCCTGCCATTCTGAAAGGCTGGTTTGAGCGAGTGTTCATAGGAGAGTTTGCTTAC
ACTTACGCTGCCATGTATGACAAAGGACCCTTCCGGAGTAAGAAGGCAGTGCTTTC
CATCACCACTGGTGGCAGTGGCTCCATGTACTCTCTGCAAGGGATCCACGGGGAC
ATGAATGTCATTCTCTGGCCAATTCAGAGTGGCATTCTGCATTTCTGTGGCTTCCAA
GTCTTAGAACCTCAACTGACATATAGCATTGGGCACACTCCAGCAGACGCCCGAAT
TCAAATCCTGGAAGGATGGAAGAAACGCCTGGAGAATATTTGGGATGAGACACCA
CTGTATTTTGCTCCAAGCAGCCTCTTTGACCTAAACTTCCAGGCAGGATTCTTAATG
AAAAAAGAGGTACAGGATGAGGAGAAAAACAAGAAATTTGGCCTTTCTGTGGGCCA
TCACTTGGGCAAGTCCATCCCAACTGACAACCAGATCAAAGCTAGAAAATGA

The NQO1 gene of the present disclosure is a member of the NAD(P)H dehydrogenase (quinone) family and encodes a cytoplasmic 2-electron reductase. Mutations in this gene are known to be associated with increased risk of hematotoxicity following benzene exposure, increased susceptibility to various types of cancer, and tardive dyskinesia (TD). The most extensively studied polymorphism of NQO1, designated as NQO1*2, involves a transition from C to T at nucleotide position 609 (609C>T; c559C>T; rs1800566) in exon 6, leading to a proline-to-serine substitution at codon 187. Individuals carrying this NQO1*2 allele can be classified into heterozygous carriers (CT type) and homozygous carriers (TT type).

The 1,2-naphthoquinone derivative has exhibited a significantly enhanced preventive or therapeutic effect in patients with mitochondrial dysfunction-related diseases who possess the wild-type allele of NQO1 (CC type) or the heterozygous NQO1*2 allele (CT type), as compared to homozygous NQO1*2 carriers (TT type).

The mutation burden refers to the degree of genetic mutation, and primary mitochondrial diseases (PMD) caused by mutations in nuclear DNA (nDNA) or mitochondrial DNA (mtDNA) generally manifest symptoms when the mutation burden exceeds 80%. This suggests that reducing the mutation burden below a symptomatic threshold could halt disease progression and improve symptoms.

In particular, for PMD caused by mutations in mtDNA-encoded genes, inducing mitophagy, a process that eliminates dysfunctional mitochondria, can reduce the mutation burden. The 1,2-naphthoquinone derivative, which is the active ingredient of the method of the present disclosure, exhibits a pharmacological effect by activating AMP-activated protein kinase (AMPK), which induces mitophagy. Consequently, the quinone derivative-mediated activation of mitophagy reduces the mutation burden in PMD patients, thereby exerting a therapeutic effect.

In one embodiment, the composition of the present disclosure is intended for administration to a subject having a CC genotype at position 609 of the NAD(P)H:quinone oxidoreductase 1 (NQO1) gene.

In one embodiment, the 1,2-naphthoquinone derivative is administered to a subject having an mtDNA A3243G mutation. Mitochondria possess their own mitochondrial DNA (mtDNA), which is distinct from nuclear DNA, and mtDNA encodes proteins essential for mitochondrial function. The A3243G mutation in the mitochondrial tRNA\hat{ }Leu(UUR) gene is recognized as a key biomarker for primary mitochondrial diseases, particularly due to its reported association with MELAS syndrome (George M C Janssen et al., The A3243G tRNA\hat{ }leu(UUR) mutation induces mitochondrial dysfunction and variable disease expression without dominant negative acting translational defects in complex IV subunits at UUR codons, Hum Mol Genet., 16(20):2472-81 (2007); C Mariotti et al., Genotype to phenotype correlations in mitochondrial encephalomyopathies associated with the A3243G mutation of mitochondrial DNA, J Neurol, 242(5):304-12 (1995)).

The mtDNA A3243G mutation leads to functional impairment of complex I, which can result in the onset of primary mitochondrial diseases. By administering the composition of the present disclosure to a subject with the A3243G mtDNA mutation, primary mitochondrial diseases can be effectively treated.

In one embodiment, the method of the present disclosure may exhibit synergistic efficacy when the 1,2-naphthoquinone derivative is administered to a subject pre-treated with an antioxidant or co-administered with an antioxidant, thereby enabling the establishment of an optimized therapeutic protocol for primary mitochondrial diseases.

In one embodiment, the antioxidant may be administered in an effective amount within 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, 18 hours, 12 hours, 6 hours, 3 hours, or 1 hour prior to administration of the 1,2-naphthoquinone derivative, or it may be administered concurrently with the composition.

In one specific embodiment, the composition of the present disclosure is intended for administration to a subject who has received an effective amount of an antioxidant concurrently with or within 30 days from the time of administration of the composition.

In another specific embodiment, the composition of the present disclosure is intended for administration to a subject who has received an effective amount of an antioxidant concurrently with or within 14 days from the time of administration of the composition.

In yet another specific embodiment, the composition of the present disclosure is intended for administration to a subject who has received an effective amount of an antioxidant concurrently with or within 7 days from the time of administration of the composition.

In one embodiment, the administration of the antioxidant may be performed in a single dose or multiple doses.

In one embodiment, the antioxidant may be at least one selected from the group consisting of vitamin C, vitamin E, vitamin K, α-lipoic acid, N-acetyl cysteine, coenzyme Q10, carnitine, riboflavin, thiamine, cysteamine, tocopherol, L-creatine, arginine, citrulline, niacin, and pyridoxine. More specifically, at least one antioxidant selected from the group consisting of vitamin C, vitamin E, coenzyme Q10 (also referred to as CoQ10), riboflavin, and vitamin K may be administered prior to or concurrently with administration of the composition of the present disclosure.

In one specific embodiment, riboflavin is used as the antioxidant of the present disclosure. When riboflavin is used as the antioxidant, it may preferably be administered at a molar ratio of at least 10-fold or greater relative to the active ingredient.

Specifically, the molar ratio of the active ingredient of the composition to riboflavin may preferably range from 1:10 to 1:100, and more specifically, from 1:11 to 1:100, 1:12 to 1:100, 1:13 to 1:100, 1:14 to 1:100, 1:15 to 1:100, 1:20 to 1:100, 1:25 to 1:100, 1:30 to 1:100, 1:40 to 1:100, or 1:50 to 1:100. However, these ratios are not limited thereto, and the amount of riboflavin may be increased as necessary.

In one embodiment, riboflavin as an antioxidant may be administered in combination with the composition of the present disclosure. The co-administration may refer to either simultaneous administration or sequential administration with a predetermined time interval, wherein the predetermined time may be appropriately selected considering the dosage and administration time of the drug, the condition of the patient, and other relevant factors. When the composition of the present disclosure and riboflavin are administered simultaneously, this includes not only administration after mixing but also sequential administration that is practically continuous.

In one embodiment, the primary mitochondrial disease is at least one selected from the group consisting of MELAS syndrome, Leigh syndrome, Kearns-Sayre syndrome, MERRF syndrome, MIDD, CPEO, and PMM.

As used herein, MELAS syndrome refers to mitochondrial encephalopathy characterized by recurrent stroke-like episodes, epilepsy, and migraines.

As used herein, Leigh syndrome refers to subacute necrotizing encephalomyelopathy, a neurodegenerative disorder affecting the brain and spinal cord, which primarily manifests in infancy. It is characterized by delayed or lost mental and motor development, ataxia, ophthalmoplegia, nystagmus, quadriplegia, hypotonia, abnormal breathing patterns, and various other clinical symptoms.

As used herein, Kearns-Sayre syndrome refers to an extremely rare mitochondrial disorder caused by mitochondrial DNA abnormalities, characterized by progressive external ophthalmoplegia (PEO) leading to ptosis, nonspecific pigmentary retinopathy, and cardiac conduction defects.

As used herein, MERRF syndrome (Myoclonic Epilepsy with Ragged-Red Fibers) is a relatively rare mitochondrial encephalomyopathy. Clinically, it manifests as progressive myoclonus, epilepsy, ataxia, limb weakness, and dementia. Histopathologically, it is characterized by the presence of “ragged-red fibers (RRF)” in muscle biopsy specimens stained using modified Gomori trichrome staining.

As used herein, MIDD (Maternally Inherited Diabetes and Deafness) is a disorder known to be caused by mitochondrial DNA variations or mutations, with the most common mutation being 3243A>G. As a mitochondrial-related disease, MIDD exhibits maternal inheritance characteristics.

As used herein, CPEO (Chronic Progressive External Ophthalmoplegia) is one of the common phenotypes of mitochondrial myopathy, caused by single deletions, multiple deletions, point mutations in mitochondrial DNA (mtDNA), or defects in nuclear-encoded mitochondrial genes.

As used herein, LHON (Leber Hereditary Optic Neuropathy) refers to a mitochondrial genetic disorder that results in painless, progressive bilateral vision loss due to optic nerve damage, typically in young adults. This disease primarily affects males (approximately 80%), whereas females tend to have a later onset and exhibit more severe symptoms.

As used herein, PMM (Primary Mitochondrial Myopathy) refers to a rare genetic disorder that can occur at any age due to mitochondrial dysfunction-induced myopathy. It is characterized by ptosis due to paralysis of the extraocular muscles, diplopia, facial paralysis, dyspnea, and motor dysfunction.

The aforementioned diseases are all classified as primary mitochondrial diseases, and they can be effectively treated using the pharmaceutical composition of the present disclosure.

In one embodiment, the pharmaceutical composition of the present disclosure comprises a pharmaceutically acceptable carrier in addition to the active ingredient. The pharmaceutically acceptable carrier included in the pharmaceutical composition may be conventionally used in formulation and may include, but is not limited to, lactose, dextrose, sucrose, sorbitol, mannitol, starch, acacia gum, calcium phosphate, alginates, gelatin, calcium silicate, microcrystalline cellulose, polyvinylpyrrolidone, cellulose, water, syrup, methylcellulose, methylparaben, propylparaben, talc, magnesium stearate, and mineral oil. The pharmaceutical composition of the present disclosure may further include lubricants, humectants, sweeteners, flavoring agents, emulsifiers, suspending agents, preservatives, and the like. Suitable pharmaceutically acceptable carriers and formulations are described in Remington's Pharmaceutical Sciences (19th ed., 1995) in detail.

The appropriate dosage of the pharmaceutical composition of the present disclosure varies depending on factors such as formulation method, mode of administration, patient's age, weight, sex, pathological condition, diet, administration time, route of administration, excretion rate, and responsiveness.

Preferably, the daily dosage of the pharmaceutical composition of the present disclosure ranges from 0.0001 mg/kg to 1000 mg/kg of body weight, including but not limited to: 0.0001 mg/kg, 0.0005 mg/kg, 0.001 mg/kg, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.2 mg/kg, 0.3 mg/kg, 0.4 mg/kg, 0.5 mg/kg, 1 mg/kg, 1.5 mg/kg, 2 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, or 1000 mg/kg.

More specifically, the dosage may be appropriately selected within the range of: 0.001-1000 mg/kg, 0.001-500 mg/kg, 0.001-100 mg/kg, 0.005-100 mg/kg, 0.01-100 mg/kg, 0.01-50 mg/kg, 0.01-40 mg/kg, 0.01-30 mg/kg, 0.01-20 mg/kg, 0.01-10 mg/kg, or 0.05-10 mg/kg, but is not limited thereto.

The pharmaceutical composition of the present disclosure may be administered orally or parenterally. When administered parenterally, it may be applied via topical application to the skin, intravenous injection, subcutaneous injection, intramuscular injection, intraperitoneal injection, or transdermal administration. Given that the pharmaceutical composition of the present disclosure is intended for the treatment of primary mitochondrial diseases, oral administration or intravenous injection is preferable.

When the composition of the present disclosure is administered orally, the term “conventional dosage form” refers to oral dosage forms (e.g., tablets, capsules, powders), as well as formulations for administration via the oral cavity, sublingual route, rectal route, vaginal route, nasal route, topical application, or parenteral administration (including intravenous, intracavernous, intramuscular, subcutaneous, and intra-arterial administration). For example, in one embodiment, the compound according to the present disclosure may be administered orally, via the oral cavity, or via the sublingual route in the form of a tablet containing starch or lactose, a capsule containing the compound alone or with excipients, or an elixir or suspension containing flavoring agents or coloring agents. Liquid formulations may be prepared as suspensions using pharmaceutically acceptable additives, such as methylcellulose, witepsol (a semi-synthetic glyceride), a mixture of apricot kernel oil and PEG-6 esters, or a mixture of PEG-8 and caprylic/capric glycerides. For parenteral administration, including intravenous, intracavernous, intramuscular, subcutaneous, or intra-arterial injection, the most preferred form is a sterile aqueous solution. The solution may contain isotonicity-adjusting agents, such as salts, mannitol, or monosaccharides like glucose, to maintain tonicity with blood.

Preferably, as described above, the pharmaceutical composition of the present disclosure may be administered orally. Solid oral formulations include tablets, pills, powders, granules, capsules, and troches, and these formulations may be prepared by mixing one or more compounds of the present disclosure with at least one excipient, such as starch, calcium carbonate, sucrose, lactose, or gelatin. Additionally, lubricants such as magnesium stearate and talc may be used in addition to simple excipients. Liquid oral formulations include suspensions, liquid preparations, emulsions, or syrups, and may contain commonly used diluents such as water or liquid paraffin, along with various excipients, including humectants, sweeteners, flavoring agents, and preservatives.

Pharmaceutical formulations for oral administration may also include sterile aqueous solutions, non-aqueous solvents, suspension agents, emulsions, lyophilized formulations, or suppositories. Non-aqueous solvents and suspension agents may include propylene glycol, polyethylene glycol, vegetable oils such as olive oil, and injectable esters such as ethyl oleate. Suppository bases may include witepsol, macrogol, Tween 61, cocoa butter, laurin butter, glycerol, and gelatin.

In one embodiment, beta-lapachone of the present disclosure may be used in the form of a pharmaceutically acceptable salt. The salt may be an acid addition salt formed by a pharmaceutically acceptable free acid. Examples of acid addition salts include salts derived from inorganic acids such as hydrochloric acid, nitric acid, phosphoric acid, sulfuric acid, hydrobromic acid, hydroiodic acid, nitrous acid, or hypophosphorous acid. Additionally, the salts may be obtained from non-toxic organic acids, such as aliphatic mono- and dicarboxylates, phenyl-substituted alkanoates, hydroxyalkanoates, alkandioates, aromatic acids, aliphatic and aromatic sulfonic acids, including acetic acid, benzoic acid, citric acid, lactic acid, maleic acid, gluconic acid, methanesulfonic acid, p-toluenesulfonic acid, tartaric acid, and fumaric acid. Other pharmaceutically acceptable non-toxic salts include sulfates, pyrosulfates, bisulfates, sulfites, bisulfites, nitrates, phosphates, monohydrogen phosphates, dihydrogen phosphates, metaphosphates, pyrophosphates, chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butene-1,4-dioates, hexane-1,6-dioates, benzoates, chlorobenzoates, methylbenzoates, dinitrobenzoates, hydroxybenzoates, methoxybenzoates, phthalates, terephthalates, benzene sulfonates, toluene sulfonates, chlorobenzene sulfonates, xylene sulfonates, phenylacetates, phenylpropionates, phenylbutyrates, citrates, lactates, p-hydroxybutyrates, glycolates, malates, tartrates, methanesulfonates, propanesulfonates, naphthalene-1-sulfonates, naphthalene-2-sulfonates, or mandelates, but are not limited thereto.

In one embodiment, the acid addition salts of the present disclosure may be prepared by conventional methods. For example, one or more 1,2-naphthoquinone derivatives represented by Chemical Formulae 1 to 3 may be dissolved in an organic solvent, such as methanol, ethanol, acetone, methylene chloride, or acetonitrile, followed by the addition of an organic or inorganic acid to form a precipitate, which is then filtered and dried. Alternatively, the solvent may be removed under reduced pressure in the presence of an excess amount of acid, followed by drying or crystallization in an organic solvent.

Furthermore, pharmaceutically acceptable metal salts may be prepared using a base. Alkali metal or alkaline earth metal salts may be obtained by dissolving the compound in an excess of an alkali metal hydroxide or alkaline earth metal hydroxide solution, filtering the resulting insoluble compound salt, and evaporating and drying the filtrate. Sodium, potassium, or calcium salts are particularly suitable for pharmaceutical applications. Additionally, the corresponding silver salts may be obtained by reacting an alkali metal or alkaline earth metal salt with an appropriate anion (e.g., silver nitrate). The present disclosure encompasses various 1,2-naphthoquinone derivatives and their pharmaceutically acceptable salts, including, but not limited to, one or more compounds represented by Chemical Formulae 1 to 3 and their pharmaceutically acceptable salts, as well as possible solvates, hydrates, and stereoisomers that may be derived therefrom.

The pharmaceutical composition of the present disclosure may be formulated into unit dosage forms or incorporated into multi-dose containers using pharmaceutically acceptable carriers and/or excipients according to methods that can be readily implemented by those skilled in the art.

In another aspect, the present disclosure provides a method for selecting a subject for administration of the aforementioned pharmaceutical composition, the method comprising determining whether a subject suffering from primary mitochondrial disease satisfies at least one of the following conditions:
(a) The genotype at position 609 of the NQO1 gene of the subject is CC or CT;
(b) Complex III, complex IV, and complex V of the mitochondrial electron transport chain in cells isolated from the subject function without dysfunction.

Satisfaction of at least one of the above conditions (a) and (b) is sufficient, and it is not necessary to satisfy both conditions simultaneously. The procedure for determining whether a subject meets these conditions is not limited to a specific method and may be performed using conventionally known techniques in the art. For example, the determination may be conducted using genetic tests such as PCR or gene sequencing or by performing activity analysis assays for mitochondrial complexes.

By implementing the method for selecting a subject for administration in the present disclosure, a more appropriate treatment protocol can be selected for a subject suffering from primary mitochondrial disease, thereby maximizing therapeutic efficacy.

The subject selection method as an aspect of the present disclosure is directed toward identifying subjects who are likely to exhibit significantly enhanced therapeutic effects upon administration of the pharmaceutical composition, which is another aspect of the present disclosure. The overlapping contents are incorporated by reference, and redundant descriptions are omitted to avoid excessive complexity in this specification.

In another aspect, the present disclosure provides a method for providing information for predicting prognosis based on the administration of the aforementioned pharmaceutical composition, the method comprising determining whether a subject suffering from primary mitochondrial disease satisfies at least one of the following conditions:
(a) The genotype at position 609 of the NQO1 gene of the subject is CC or CT;
(b) Complex III, complex IV, and complex V of the mitochondrial electron transport chain in cells isolated from the subject function without dysfunction;

If at least one of the above conditions is met, the prognosis following administration of the pharmaceutical composition is predicted to be favorable.

Satisfaction of at least one of conditions (a) and (b) is sufficient, and it is not necessary to satisfy both conditions simultaneously. The procedure for determining whether a subject meets these conditions is not limited to a specific method and may be performed using conventionally known techniques in the art. For example, the determination may be conducted using genetic tests such as PCR or gene sequencing or by performing activity analysis assays for mitochondrial complexes.

The method for selecting a subject for administration, as an aspect of the present disclosure, relates to selecting a subject who is expected to have a favorable prognosis upon administration of the pharmaceutical composition, which is another aspect of the present disclosure. The overlapping contents are incorporated by reference, and redundant descriptions are omitted to avoid excessive complexity in this specification.

In another aspect, the present disclosure provides a functional food composition comprising a 1,2-naphthoquinone derivative and exhibiting at least one of the following functionalities:
(a) Increasing total ATP levels in the body;
(b) Reducing total lactate levels in the body; or
(c) Reducing total ROS levels in the body.

The functional food composition of the present disclosure functions to restore or activate mitochondrial electron transport chain activity, thereby effectively achieving the effects of increasing ATP levels and reducing total lactate and ROS levels in the body.

In one embodiment, the composition of the present disclosure has a preventive or ameliorative effect on primary mitochondrial diseases.

The functional food composition of the present disclosure may include ingredients commonly used in food manufacturing, such as proteins, carbohydrates, fats, nutrients, and seasonings. For example, when formulated as a drink, additional components such as flavoring agents or natural carbohydrates may be included along with the active ingredient.

Examples of natural carbohydrates include: Monosaccharides (e.g., glucose, fructose); Disaccharides (e.g., maltose, sucrose); Oligosaccharides; Polysaccharides (e.g., dextrin, cyclodextrin); and Sugar alcohols (e.g., xylitol, sorbitol, erythritol).

Examples of flavoring agents include: Natural flavoring agents (e.g., thaumatin, stevia extract); and

Synthetic flavoring agents (e.g., saccharin, aspartame).

The functional food composition, as an aspect of the present disclosure, comprises the same active ingredient as the pharmaceutical composition, which is another aspect of the present disclosure. The overlapping contents are incorporated by reference, and redundant descriptions are omitted to avoid excessive complexity in this specification.

### Advantageous Effects of Invention

The features and advantages of the present disclosure are summarized as follows:
(a) The present disclosure provides a pharmaceutical composition for the prevention or treatment of primary mitochondrial diseases.
(b) Another aspect of the present disclosure provides a method for selecting a subject for administration of the composition.
(c) Yet another aspect of the present disclosure provides a method for providing information to predict prognosis based on the administration of the composition.
(d) By utilizing the present disclosure, subjects can be effectively selected for drug administration, enabling targeted administration of the composition and achieving high-efficiency prevention or treatment of primary mitochondrial diseases.

### BRIEF DESCRIPTION OF DRAWINGS

FIG. 1 illustrates the results of NQO1 activity testing following the administration of a 1,2-naphthoquinone derivative.

FIG. 2 illustrates the results of lactate level measurements following the administration of a 1,2-naphthoquinone derivative.

FIG. 3 illustrates the analysis of ATP level changes based on NQO1 genotype in Leigh patient-derived cells.

FIG. 4 illustrates the analysis of lactate level changes based on NQO1 genotype in Leigh patient-derived cells.

FIG. 5 illustrates the analysis of ROS level changes based on NQO1 genotype in Leigh patient-derived cells.

FIG. 6 illustrates the therapeutic efficacy of beta-lapachone against LHON disease, as evaluated using an LHON-induced animal model.

FIG. 7 illustrates the results of lactate level measurements in MELAS cell lines treated with beta-lapachone, dunnione, the compound of Chemical Formula 3 in this disclosure, and idebenone, to compare the efficacy of NQO1-targeting compounds.

FIG. 8 illustrates the effect of beta-lapachone on ATP increase in an NQO1-dependent manner, confirmed in cells deficient in mitochondrial complexes I and II.

FIG. 9 illustrates the results of combination treatment with an antioxidant in MELAS cell lines, evaluating its synergistic therapeutic efficacy.

### DETAILED DESCRIPTION

Hereinafter, the present disclosure will be described in further detail through the following Examples. These Examples are provided solely for the purpose of illustrating the present disclosure in greater detail and should not be construed as limiting the scope of the present disclosure. It will be apparent to those skilled in the art that the scope of the present disclosure is not restricted to these Examples but is defined by the claims and their equivalents.

### Examples

### Example 1: Analysis of NQO1 Activity in Human Recombinant NQO1 Enzyme by Evaluating Cytochrome C Reduction

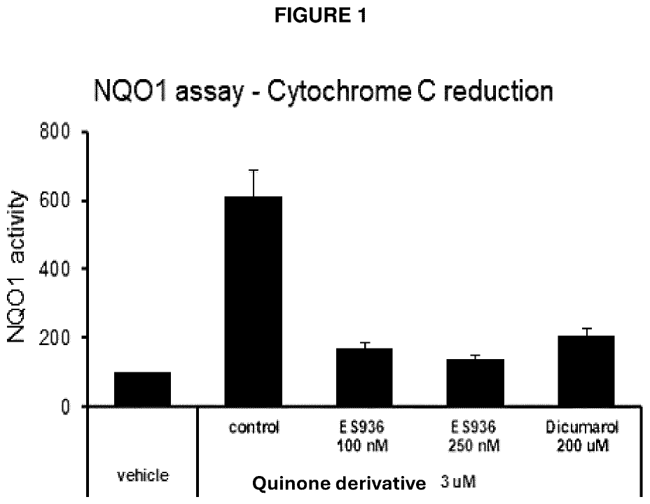

To analyze NQO1 enzyme activity, 2.5 mU of human NQO1 recombinant protein (Human NQO1 recombinant Protein, Ab59663, Abcam, Eppelheim, Germany) was treated with cytochrome C and beta-lapachone as a quinone derivative in a 50 mM Tris-HCl aqueous solution containing 0.14% BSA. The reaction was monitored using absorbance at 550 nm, which allows the measurement of cytochrome C reduction, and the results are shown in FIG. 1. As shown in FIG. 1, the quinone derivative beta-lapachone exhibited higher NQO1 activity compared to the vehicle-treated control group. However, when the NQO1 inhibitors ES936 and dicoumarol were administered along with the quinone derivative, NQO1 activity was almost completely inhibited.

NQO1 can be classified into CC (wild-type allele), CT (heterozygous allele), and TT (mutant allele) genotypes based on the C609T polymorphism. Among these, individuals carrying the T allele undergo a proline-to-serine substitution at position 187, leading to structural instability of NQO1, rapid protein degradation, and ultimately, a loss of NQO1 enzymatic activity (Relative NQO1 activity by genotype: CC>CT>TT). In FIG. 1, the control group represents a model with fully functional NQO1 activity, corresponding to a CC-like model. In contrast, the groups treated with NQO1 inhibitors ES936 and dicoumarol, which exhibit no detectable NQO1 activity, correspond to a TT-like model.

### Example 2: Analysis of Total Lactate Levels in MELAS Cell Lines

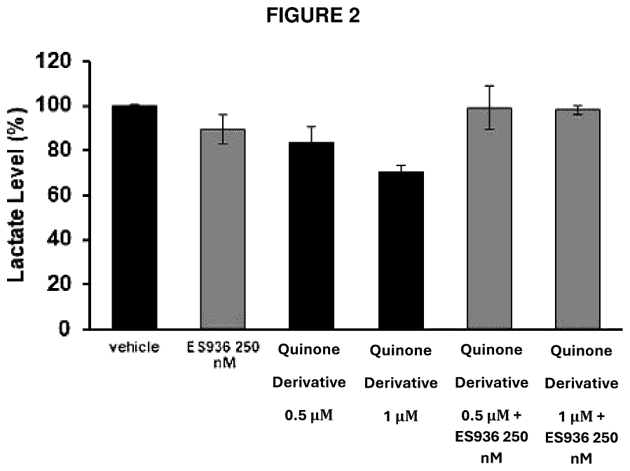

The effect of beta-lapachone, included in the composition of the present disclosure, on total lactate levels was investigated. Total intracellular lactate levels were measured using a Lactate Assay Kit (MET-5012, Cell Biolabs, San Diego, CA, USA). MELAS cell lines were used for the analysis, and the quinone derivative-treated experimental group was compared to the vehicle-treated control group. Each compound was administered at concentrations of 0.5 μM and 1 μM, and to confirm NQO1 dependency, the NQO1 inhibitor ES936 was co-administered. After treating the cells with each compound for 24 hours, absorbance at 570 nm was measured using a spectrophotometer, and the total lactate levels were determined. The results are shown in FIG. 2.

As shown in FIG. 2, beta-lapachone reduced total lactate levels compared to the vehicle-treated control group. This effect was completely inhibited by co-administration of ES936, confirming NQO1 dependency.

Based on these results, the quinone derivatives of the present disclosure exhibit excellent mitochondrial function enhancement and antioxidant effects, demonstrating their effective applicability in the prevention and treatment of various mitochondrial dysfunction-related diseases, including MELAS syndrome. In particular, it was confirmed that in subjects with the wild-type NQO1 allele (CC type), the quinone derivatives are highly effective in addressing mitochondrial dysfunction. However, in homozygous mutant (TT) patients, the preventive, ameliorative, or therapeutic effects on the disease may be relatively lower.

### Example 3: Analysis of ATP Level Changes Based on NQO1 Genotype in Leigh Syndrome Patient Cells

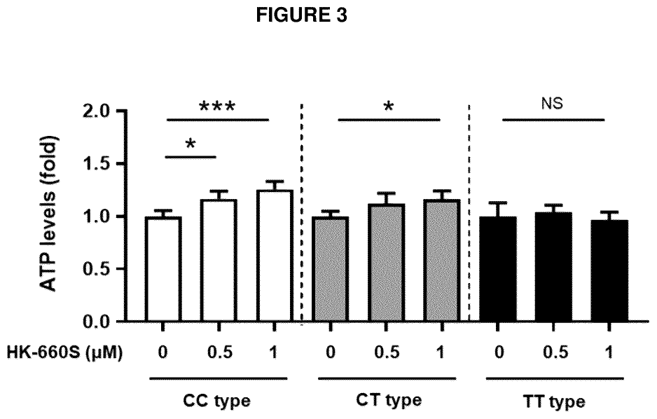

In previous experiments, the efficacy of the quinone derivative beta-lapachone was confirmed based on NQO1 genotype in MELAS syndrome patient cells. To further investigate its efficacy in other mitochondrial diseases, the effect of beta-lapachone on total ATP levels was examined using Leigh syndrome patient cells. Patient-derived cells with CC, CT, and TT genotypes were treated with beta-lapachone at 0.5 μM, 1 μM, and 2 μM, followed by 24-hour incubation. The total ATP levels were measured using the ATPlite 1 Step Luminescence Assay System (6016736, PerkinElmer, Waltham, MA, USA), and the results were analyzed using a luminometer (FIG. 3). As shown in FIG. 3, beta-lapachone significantly increased ATP levels in cells expressing NQO1 with CC and CT genotypes. However, in cells expressing NQO1 with the TT genotype, no significant effect was observed.

### Example 4: Analysis of Lactate Level Changes Based on NQO1 Genotype in Leigh Syndrome Patient Cells

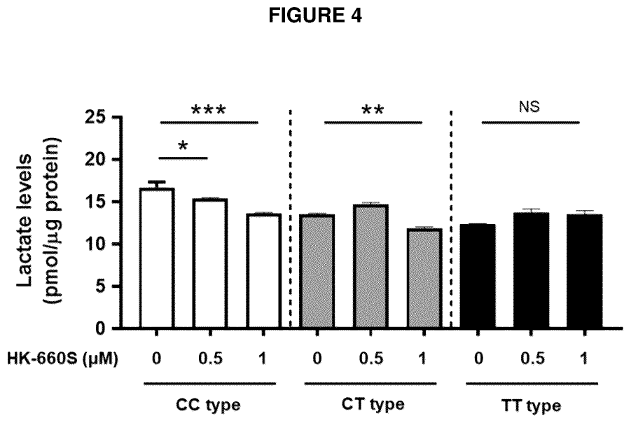

Next, the effect of beta-lapachone on total lactate levels was examined in Leigh syndrome patient-derived cells. Patient cells expressing NQO1 with CC, CT, and TT genotypes were treated with beta-lapachone at 0.5 μM, 1 μM, and 2 μM and incubated for 24 hours. Total intracellular lactate levels were measured using the Lactate Assay Kit (MET-5012, Cellbiolabs, San Diego, CA, USA), and absorbance at 570 nm was analyzed using a spectrophotometer (FIG. 4).

As shown in FIG. 4, beta-lapachone significantly reduced lactate levels in cells expressing NQO1 with CC and CT genotypes. However, in cells expressing NQO1 with the TT genotype, no significant effect was observed.

### Example 5: Analysis of ROS Level Changes Based on NQO1 Genotype in Leigh Syndrome Patient Cells

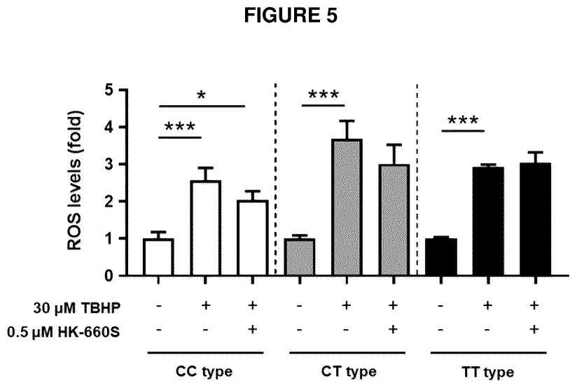

Next, the effect of beta-lapachone on total ROS levels was examined in Leigh syndrome patient-derived cells. Patient cells expressing NQO1 with CC, CT, and TT genotypes were treated with beta-lapachone at 0.5 μM, 1 μM, and 2 μM and incubated for 24 hours. Total intracellular ROS levels were measured using 5 μM CM-H₂DCFDA (C6827, Invitrogen, Eugene, OR, USA). After staining at 37° C. for 30 minutes, fluorescence intensity (Ex/Em: 495/520 nm) was analyzed using a flow cytometer (FIG. 5).

As shown in FIG. 5, beta-lapachone significantly reduced ROS levels in cells expressing NQO1 with CC and CT genotypes. However, in cells expressing NQO1 with the TT genotype, no significant effect was observed.

### Example 6: Validation of Beta-Lapachone Efficacy in an LHON-Induced Animal Model

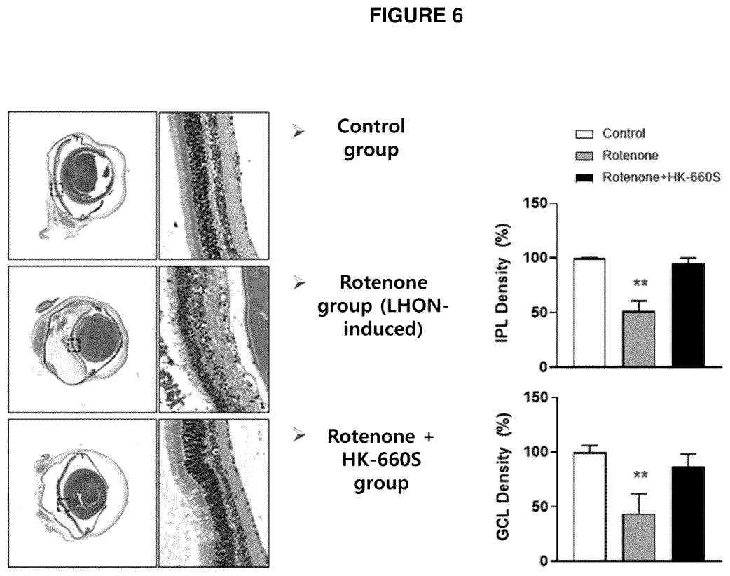

To evaluate the efficacy of beta-lapachone in an LHON-induced animal model, rotenone was directly injected into the eyes of mice to induce LHON-like characteristics in the animal model. To assess the therapeutic effects of beta-lapachone, LHON-induced animals were orally administered beta-lapachone at 80 mpk for 7 days. After 7 days, the animals were sacrificed, and their eyes were extracted for histological staining. The density of the ganglion cell layer (GCL), which contains densely packed ganglion cells, and the inner plexiform layer (IPL), where neuronal synapses are formed, was quantitatively analyzed (FIG. 6).

As shown in FIG. 6, in the rotenone-induced LHON group, the GCL density was 43.89% compared to the normal control group, showing a significant reduction. However, in the beta-lapachone-treated LHON group, the GCL density was 87.08%, which was significantly higher than the rotenone-induced LHON group. Similarly, the IPL density in the rotenone-induced LHON group was 51.31% compared to the normal control group. In contrast, in the beta-lapachone-treated LHON group, the IPL density reached 94.58%, indicating a substantial recovery.

These results demonstrate that beta-lapachone exhibits excellent therapeutic effects for LHON, suggesting its potential for treating LHON-related neurodegeneration.

### Example 7: Comparative Analysis of the Efficacy of NQO1-Targeting Compounds

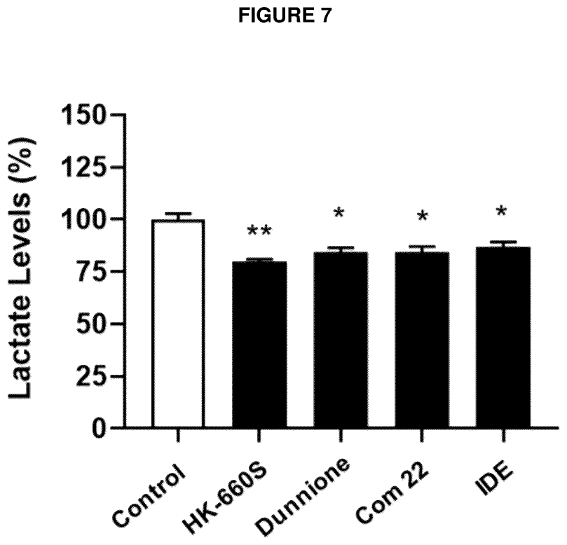

Based on previous findings demonstrating the therapeutic effects of the quinone derivative beta-lapachone on mitochondrial diseases, a comparative efficacy study was conducted to identify the most optimal compound among NQO1-targeting quinone derivatives. To this end, MELAS cell lines were treated with 1 μM of beta-lapachone, dunnione, the compound of Chemical Formula 3, and idebenone for 24 hours, after which total intracellular lactate levels were measured (FIG. 7).

As shown in FIG. 7, all tested compounds significantly reduced total lactate levels compared to the control group. However, beta-lapachone exhibited the most potent effect, showing: 1.3-fold greater reduction compared to dunnione; 1.3-fold greater reduction compared to the compound of Chemical Formula 3; and 1.5-fold greater reduction compared to idebenone.

These results indicate that, among the quinone derivatives evaluated in the present disclosure, beta-lapachone exhibits the highest efficacy in mitochondrial disease cell models, highlighting its potential as an optimal therapeutic candidate.

### Example 8: ATP Enhancement Effect in Cells Deficient in Mitochondrial Complex I and II

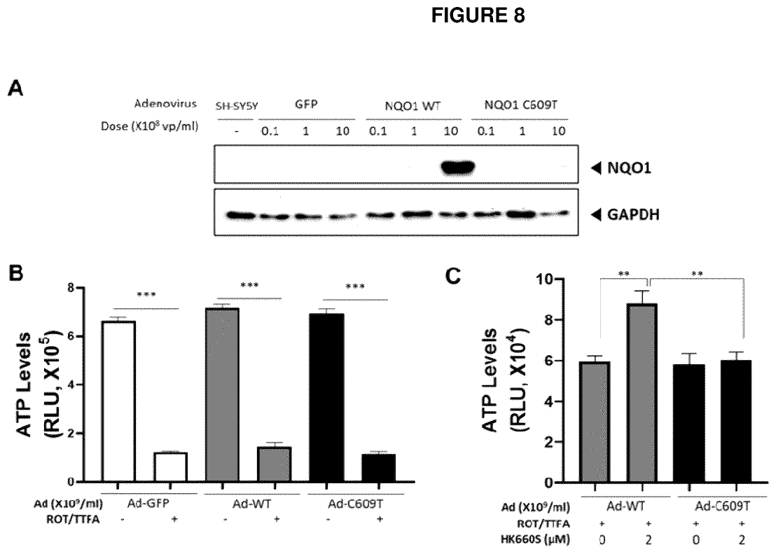

To determine whether beta-lapachone induces ATP increase in an NQO1-dependent manner in neuronal cells with suppressed mitochondrial complex I and II activity, an experiment was conducted using SH-SY5Y cells. SH-SY5Y cells were transduced with adenoviruses expressing either wild-type NQO1 (CC type, WT) or mutant NQO1 (C609T mutation, TT type) for 24 hours. As shown in FIG. 8A, compared to cells expressing only GFP (control group), NQO1 protein expression was significantly increased in NQO1 WT-expressing cells, whereas NQO1 C609T-expressing cells exhibited minimal protein expression due to low protein stability. Next, after adenovirus transduction, SH-SY5Y cells were treated with rotenone (a complex I inhibitor) and TTFA (a complex II inhibitor). The resulting ATP depletion was similar among the GFP control, NQO1 WT, and NQO1 C609T groups (FIG. 8B). Following this, the same cells were treated with 2 μM beta-lapachone for 10 minutes. In NQO1 WT-expressing cells, ATP levels increased significantly by approximately 50%, whereas no ATP increase was observed in NQO1 C609T-expressing cells (FIG. 8C).

These findings demonstrate that even in the absence of mitochondrial complex I and II activity, beta-lapachone effectively restores ATP levels, confirming that this effect is NQO1-dependent.

### Example 9: Synergistic Effect of Beta-Lapachone and Antioxidants in MELAS Cell Lines

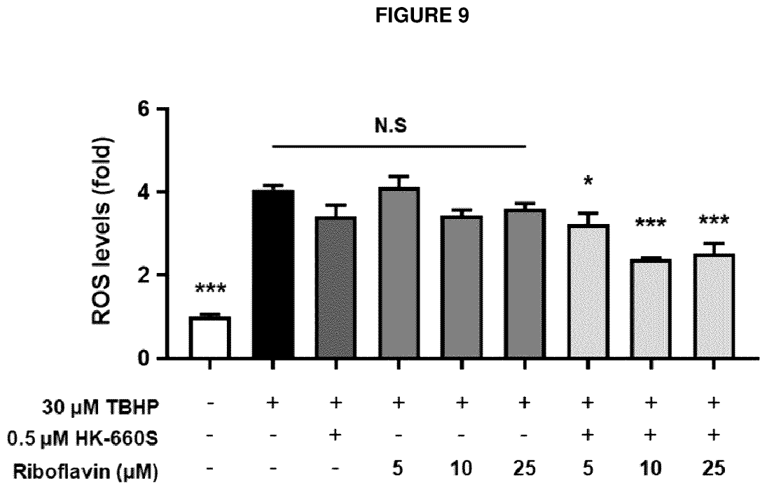

To investigate whether the combination of beta-lapachone and an antioxidant commonly used for PMD patients effectively reduces ROS levels, experiments were conducted using MELAS cell lines. MELAS cells were treated for 24 hours with 0.5 μM beta-lapachone, and either 5 μM, 10 μM, or 25 μM of riboflavin alone or in combination. To induce ROS production, 30 μM TBHP (tert-butyl hydroperoxide) was added 3 hours before the experiment ended, and the effects of each condition on ROS levels were evaluated (FIG. 9). Total intracellular ROS levels were measured using 5 μM CM-H₂DCFDA (C6827, Invitrogen, Eugene, OR, USA). After staining at 37° C. for 30 minutes, fluorescence intensity (Ex/Em: 495/520 nm) was analyzed using a flow cytometer.

The results showed that a decreasing trend in ROS levels was observed in the groups treated with 0.5 μM beta-lapachone and either 10 μM or 25 μM riboflavin, but these reductions were not statistically significant. The 5 μM riboflavin monotherapy group did not exhibit any ROS reduction. However, in the beta-lapachone and riboflavin combination-treated groups, a significant reduction in ROS levels was observed compared to the TBHP-treated control group. These findings indicate that the co-administration of beta-lapachone and an antioxidant, such as riboflavin, may provide a synergistic effect in reducing ROS levels, suggesting that combination therapy may be beneficial for MELAS patients.

What is claimed is:

1. A method of treating a primary mitochondrial disease in a subject in need thereof, the method comprising:
administering to the subject a pharmaceutically effective amount of a 1,2-naphthoquinone derivative.

2. The method according to claim 1, wherein the 1,2-naphthoquinone derivative is at least one compound selected from the group consisting of compounds represented by Chemical Formulae 1 to 3:

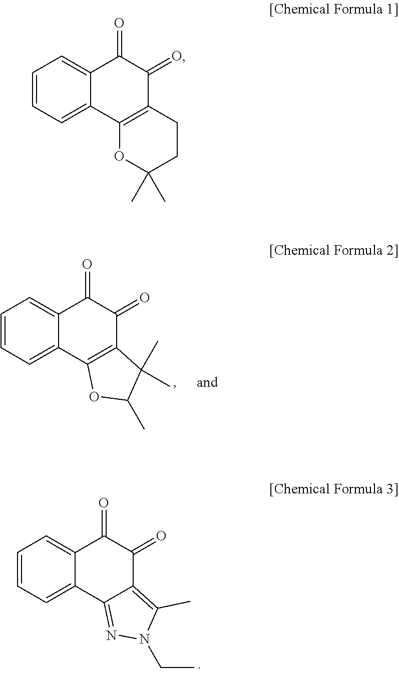 [SMILES: CC1(C)CCC2=C(O1)c1ccccc1C(=O)C2=O ; CC1OC2=C(C(=O)C(=O)c3ccccc32)C1(C)C ; CCn1nc2c(c1C)C(=O)C(=O)c1ccccc1-2]

3. The method according to claim 1, wherein the 1,2-naphthoquinone derivative is administered to a subject in whom complex III, complex IV, and complex V of the mitochondrial electron transport chain function normally.

4. The method according to claim 1, wherein the 1,2-naphthoquinone derivative is administered to a subject with impaired complex I of the mitochondrial electron transport chain.

5. The method according to claim 1, wherein the 1,2-naphthoquinone derivative is administered to a subject having a genotype of CC or CT at position 609 of the NAD(P)H:quinone oxidoreductase 1 (NQO1) gene.

6. The method according to claim 5, wherein the 1,2-naphthoquinone derivative is administered to a subject having a genotype of CC at position 609 of the NAD(P)H:quinone oxidoreductase 1 (NQO1) gene.

7. The method according to claim 1, wherein the 1,2-naphthoquinone derivative is administered to a subject having an mtDNA A3243G mutation.

8. The method according to claim 1, wherein the 1,2-naphthoquinone derivative is administered to a subject who has been administered an effective amount of an antioxidant concurrently or within 30 days from the time of administration of the composition.

9. The method according to claim 8, wherein the 1,2-naphthoquinone derivative is administered to a subject who has been administered an effective amount of an antioxidant concurrently or within 14 days from the time of administration of the 1,2-naphthoquinone derivative.

10. The method according to claim 8, wherein the 1,2-naphthoquinone derivative is administered to a subject who has been administered an effective amount of an antioxidant concurrently or within 7 days from the time of administration of the 1,2-naphthoquinone derivative.

11. The method according to claim 8, wherein the antioxidant is administered in a single dose or in multiple doses.

12. The method according to claim 8, wherein the antioxidant is at least one selected from the group consisting of vitamin C, vitamin E, vitamin K, α-lipoic acid, N-acetyl cysteine, coenzyme Q10, carnitine, riboflavin, thiamine, cysteamine, tocopherol, L-creatine, arginine, citrulline, niacin, and pyridoxine.

13. The method according to claim 12, wherein the antioxidant is riboflavin.

14. The method according to claim 1, wherein the primary mitochondrial disease is at least one selected from the group consisting of MELAS syndrome (mitochondrial encephalopathy, lactic acidosis, and stroke-like episodes), Leigh syndrome, Kearns-Sayre syndrome, MERRF (myoclonus epilepsy with ragged-red fibers), MIDD (maternally inherited diabetes and deafness), CPEO (chronic progressive external ophthalmoplegia), LHON (Leber hereditary optic neuropathy), and PMM (primary mitochondrial myopathy).

15. A method of selecting a subject for administration of 1,2-naphthoquinone derivative, comprising:
determining whether the subject suffering from primary mitochondrial disease meets one or more of the following conditions:
(a) the genotype at position 609 of the NQO1 gene of the subject is CC or CT; and
(b) complex III, complex IV, and complex V of the mitochondrial electron transport chain in cells isolated from the subject operate normally without dysfunction.

16. The method according to claim 15, wherein the 1,2-naphthoquinone derivative is at least one compound selected from the group consisting of compounds represented by Chemical Formulae 1 to 3:

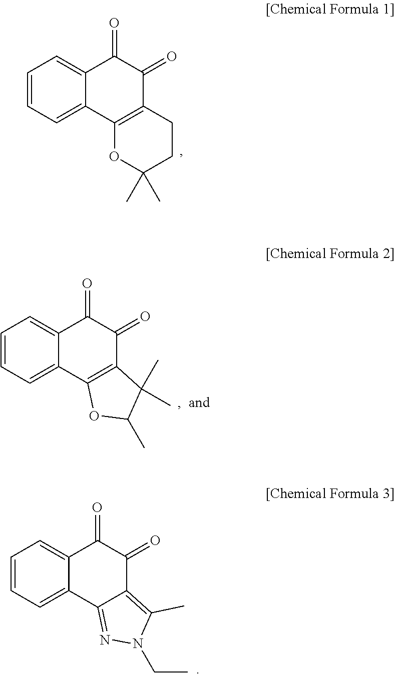 [SMILES: CC1(C)CCC2=C(O1)c1ccccc1C(=O)C2=O ; CC1OC2=C(C(=O)C(=O)c3ccccc32)C1(C)C ; CCn1nc2c(c1C)C(=O)C(=O)c1ccccc1-2]

17. A method for predicting prognosis following administration of the 1,2-naphthoquinone derivative, comprising:
determining whether the subject suffering from primary mitochondrial disease meets one or more of the following conditions:
(a) the genotype at position 609 of the NQO1 gene of the subject is CC or CT;
(b) complex III, complex IV, and complex V of the mitochondrial electron transport chain in cells isolated from the subject operate normally without dysfunction;
wherein, if one or more of the above conditions are met, the prognosis following administration of the 1,2-naphthoquinone derivative is predicted to be favorable.

18. The method according to claim 17, wherein the 1,2-naphthoquinone derivative is at least one compound selected from the group consisting of compounds represented by Chemical Formulae 1 to 3: 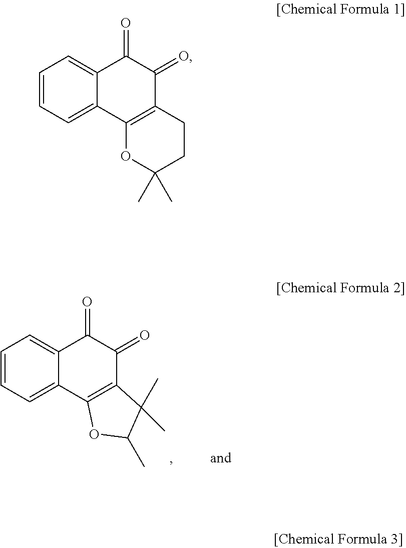 [SMILES: CC1(C)CCC2=C(O1)c1ccccc1C(=O)C2=O ; CC1OC2=C(C(=O)C(=O)c3ccccc32)C1(C)C]
